(19) United States
(12) Patent Application Publication
(10) Pub. No.: US 2005/0049703 A1
(43) Pub. Date: Mar. 3, 2005

(54) Spinal implant

(76) Inventor: Casey K. Lee, Florham Park, NJ (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 10/648,001

(22) Filed: Aug. 26, 2003

(51) Int. Cl. A61F 2/44

(52) U.S. Cl. 623/17.11

(57) ABSTRACT

Lumbar and thoracic spinal implants comprised of substantially cancellous bone and/or calcaneus are disclosed. The graft lumbar and thoracic spinal implants have sufficient load bearing capacity to withstand the loads of the lumbar and thoracic spine.

### FIELD OF THE INVENTION

The invention relates to spinal implants and methods for their preparation and use.

### BACKGROUND OF THE INVENTION

Intervertebral fusion is one of many procedures used in the treatment of degenerative and traumatic spinal disorders such as disc disease. Interbody fusion stabilizes the spine and contributes to reducing the pain associated with degenerative disc disease. Fusion procedures are most frequently performed in the lumbar spine, however, they are also performed in the cervical spine and thoracic spine. Generally, there are several types of lumbar spinal fusion procedures: posterior lumbar interbody fusion (PLIF); anterior lumbar interbody fusion (ALIF); transforaminal interbody lumbar fusion (TLIF); posterior fusion; and postero-lateral fusion.

In posterior lumbar interbody fusion, (PLIF), two adjacent vertebral bodies are fused together by removing the affected disc and inserting an implant between the vertebral bodies, maintaining the space between the bodies and facilitating fusion by creating a space where fusing bone can form. An anterior lumbar interbody fusion (ALIF) is similar to the posterior lumbar interbody fusion (PLIF), except that the spine is accessed through the abdomen instead of through the back of the patient. The third type of procedure, a transforaminal lumbar interbody fusion (TLIF), is performed through the back part of the spine. In a TLIF procedure, the anterior (front) and posterior (back) columns of the spine are fused through a single posterior approach. In the cervical spine, an anterior interbody fusion (ACIF) is achieved in a similar approach.

In fusion procedures, bone grafts can be obtained from the patient or from a tissue bank. Grafts cut from the patient are called autografts, and these autografts may be cut from various parts of the patient's body. Frequently, autografts are taken from the iliac crest of the patient. More recently, however, allografts, or graft materials obtained from tissue banks have been increasingly used in the above procedures. The use of allografts can eliminate the need to perform a painful surgery to obtain bone from the patient, reducing one source of risk and pain of the fusion surgery. The most commonly used bones used to provide allografts include bones from the pelvis, the fibula and the femur. In lumbar spine fusion procedures, femoral bone is primarily used because of the strength of the femoral bone, which is comprised of a high percentage of cortical bone. Generally, a typical femoral bone used as an allograft spinal implant comprises greater than 90% by volume of cortical bone.

Mostly cancellous allografts from the calcaneus have been used for cervical spine implants, and these mostly cancellous allografts promote faster healing compared to allografts that are comprised of a high percentage of cortical bone. It is believed, however, that mostly cancellous allografts have not been used in the lumbar or thoracic spine or thoracic spine area because cancellous bone does not have the weight bearing properties required for implants subjected to mechanical loads in these regions of the spine. According to calculations in the published literature, 10% of the total body weight is above the thoracic spine (T1), and about 50% is of the body weight is above the thoracolumbar spine (T12). Augustus A. White III and Manohar M. Panjabi, Clinical Biomechanics of the Spine, page 530 (2^{nd} ed. 1990). In the lumbar region, muscle forces impart loads to the spine to as much as three to four times body weight. Accordingly, grafts implanted in the cervical spine region encounter less severe loads than grafts implanted below the cervical spine region, that is, the thoracic spine (T1-T12), the thoracolumbar spine (T11-L1) and the lumbar and lumbosacral spine (L1-S1) because of the added forces due to dynamic loading and physiologic muscle forces.

Although a variety of allograft implants exist for the region below the cervical spine, and in particular, the lumbar spine, improved allograft implants are needed, particularly as the supply of allografts is currently limited. It would be desirable to provide spinal allograft implants that can survive the loading encountered below the cervical spine that promote faster healing and integration than cortical bone allograft implants, and have sufficient weight bearing capacity when implanted in the lumbar and lumbosacral spine.

### SUMMARY OF THE INVENTION

In accordance with one or more embodiments of the present invention, a thoracic and lumbar spinal implant are provided. As used herein, the term “thoracic spine” includes the thoracic spine region (T1-T12) and the thoracolumbar spine region (T11-L1), and the term “lumbar spine” includes the lumbar and lumbosacral spine (L1-S1). The implant preferably comprises a solid body of greater than 10% dense cancellous bone that is sized and shaped for placement between to lumbar vertebral bodies. In certain preferred embodiments, the graft comprises substantially dense cancellous bone that is sized and shaped for placement between two lumbar vertebral bodies. In preferred embodiments, the implant is an allograft. The implant is capable of bearing weight in the thoracic spine and lumbar spine of a human being. One or more embodiments relate to a thoracic or lumbar spinal implant allograft comprised of calcaneus. Another embodiment of the invention relates to a method of performing a spinal fusion on a patient comprising placing an implant comprised of greater than 10% dense cancellous bone between two thoracic or lumbar vertebral bodies. In preferred embodiments, the implant comprises an allograft of calcaneus. In still another embodiment of the invention, a method of preparing a spinal implant comprises cutting a portion of a calcaneus from a donor.

### BRIEF DESCRIPTION OF THE DRAWINGS

A more complete appreciation of the subject matter of the present invention and the various advantages thereof can be realized by reference to the following detailed description in which reference is made to the accompanying drawings in which:

FIG. 1 is a bottom plan view of the bones of a human foot; and

FIG. 2 is lateral view of the bones of a human foot;

FIG. 3 is a cross-sectional view of a section of calcaneus taken along line 3-3 of FIG. 2;

FIG. 4 is a side perspective view of the section of calcaneus shown in FIG. 3; and

FIG. 5 is a cross sectional view of an allograft implant according to one embodiment of the present invention.

### DETAILED DESCRIPTION OF EXEMPLARY EMBODIMENTS

Before describing several exemplary embodiments of the invention, it is to be understood that the invention is not limited to the details of construction or process steps set forth in the following description. The invention is capable of other embodiments and of being practiced or carried out in various ways.

In overview, one or more embodiments of the invention relates to a thoracic or lumbar spinal implant. According to certain preferred embodiments an implant comprised of greater than about 10% by volume cancellous bone is capable of supporting the weight in the lumbar spine of a patient when implanted between two vertebrae. Preferably, the cancellous bone is dense cancellous bone. In preferred embodiments, the implant is comprised of substantially cancellous bone. As used herein for one or more embodiments, the phrase “substantially cancellous bone” means bone that is comprised of greater than about 50% cancellous bone by volume. In preferred embodiments, calcaneus is used as a source of dense cancellous bone, which is greater than about 80% dense cancellous bone by volume. In highly preferred embodiments, the implants comprise than about 95% to 98% cancellous bone by volume.

In preferred embodiments, the allograft is comprised of a solid body or mass of substantially dense cancellous bone. As used herein, the phrase or term “solid body” or “mass” means at least one unitary piece of bone removed from a donor, as opposed to a graft made from chips or particles of bone formed into a single piece. It is to be understood that the implant can comprise more than one solid body or mass of bone. For example, an implant can comprise two or more pieces of bone implanted in the thoracic or lumbar spine. Thus, preferably, the implant comprises a solid body includes substantially of dense cancellous bone, a portion of which is surrounded by a thin layer of cortical bone.

In certain preferred embodiments, the cancellous bone is surrounded on at least three sides by a thin layer of cortical bone. According to one or more embodiments of the present invention, the implant is capable of bearing weight in the thoracic or lumbar spine of a human being. Preferably, the implant has a weight bearing capacity of at least about 500 pounds, or more preferably, about 1000 pounds or between about 4 to 5 kiloNewtons. As discussed above in the background section, the published literature has stated that the compressive load encountered by the lumbar spine during normal, daily activities is 3 to 4 times a person's body weight. Accordingly, for a person weighing 250 pounds, the lumbar spine may encounter about 1000 pounds of compressive load. The thoracic spine encounters more severe loads than the cervical spine.

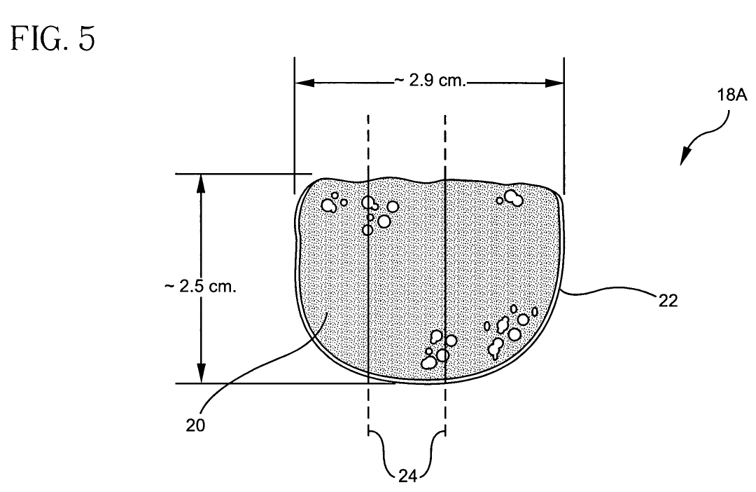

According to certain embodiments, the surface area of the implant in contact with the vertebral bodies comprises at least about 80%, and preferably at least about 98% cancellous bone. In highly preferred embodiments, the bone source comprises calcaneus or is calcis. In certain preferred embodiments, the surface area in contact between the implant and two adjacent vertebrae is at least 6 square centimeters, and more preferably 6.25 square centimeters. Suitable sizes for spinal implants in the lumbar are approximately 3 centimeters by about 2.5 centimeters in cross-section as shown in FIG. 5. The thickness of the implant can range from between about 0.5 centimeters and 5 centimeters. The thickness of the implant may vary for a single implant. It will be understood, of course, that these dimensions and the contact surface area between the implant and the vertebrae will vary depending on the needs of the patient and the particular surgery.

Another embodiment of the invention relates to a method of performing a spinal fusion on a patient comprising implanting graft comprising substantially cancellous bone between two lumbar vertebral bodies or two thoracic vertebral bodies. In preferred embodiments, the graft is an allograft, and in highly preferred embodiments, the allograft comprises a portion of a calcaneus. The graft is implanted using conventional fusion procedures such as an anterior lumbar interbody fusion (ALIF), a posterior lumbar interbody fusion (PLIF) or a transforaminal lumbar interbody fusion (TLIF).

Still another embodiment of the invention relates to a method of making an allograft lumbar spinal implant or thoracic spinal implant comprising cutting a portion of the calcaneus from a donor in a size and shape for insertion between two vertebral bodies. The donor is a cadaver, and the bone cut from the donor is frozen or freeze dried, as is known in the art.

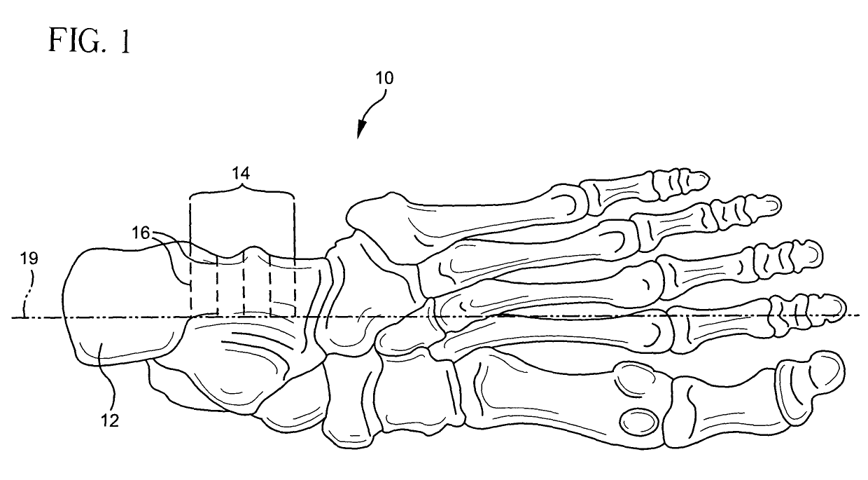

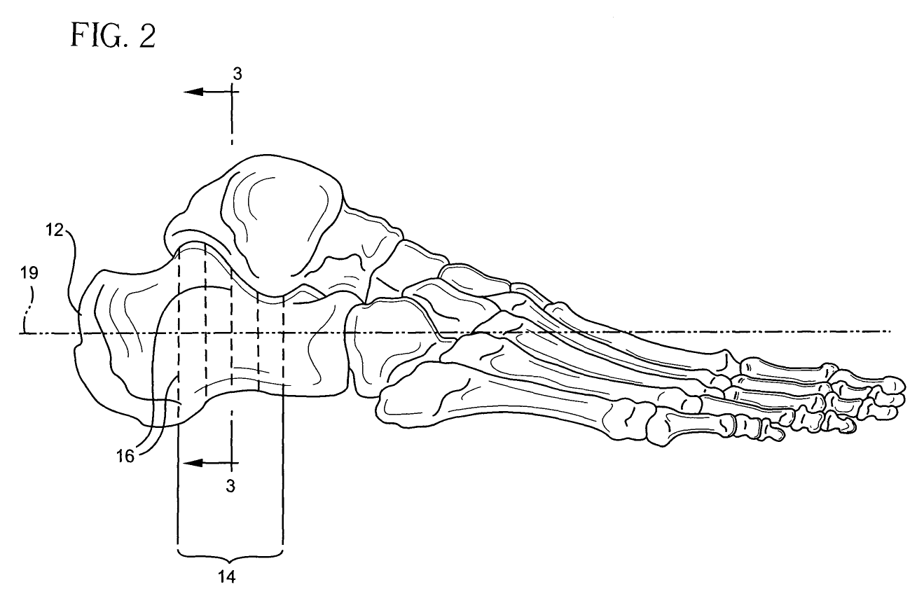

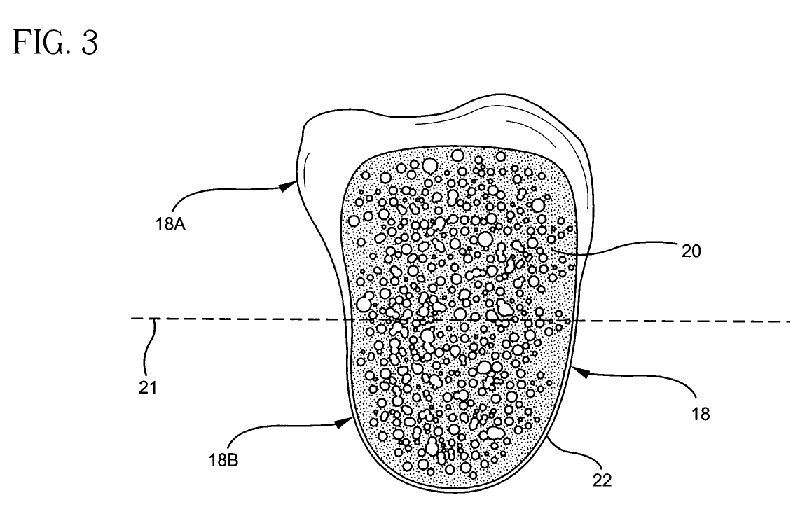

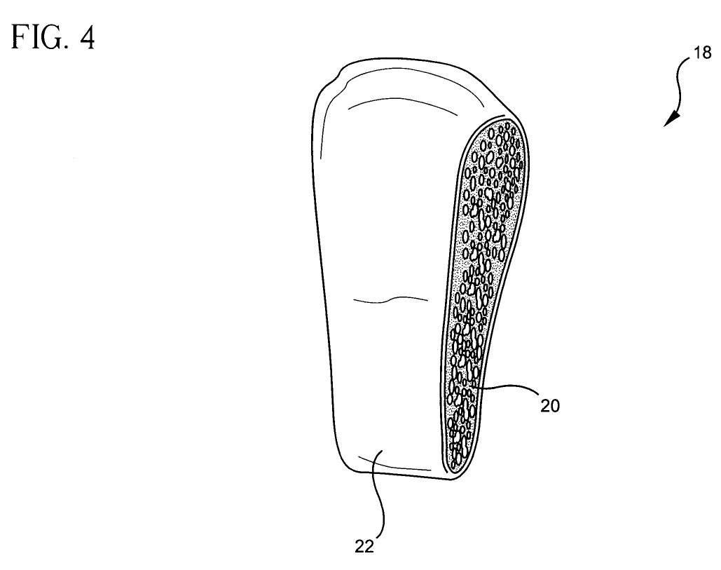

According to one or more embodiments, the invention relates to a specialized method of cutting the calcaneus from a donor. FIGS. 1 and 2 show views of the bones of a human foot 10. The calcaneus or heel bone 12 is cut using an appropriate cutting instrument such as a reciprocating saw, a sagittal saw, a burr, or a laser. In preferred embodiments, a central portion 14 of the calcaneus is cut along the lines 16 indicated in FIGS. 1 and 2. As shown in FIGS. 1 and 2, the calcaneus is preferably cut substantially perpendicular to the long axis of the bone 19. Of course, the calcaneus may be cut in a different manner, perhaps given the particular fusion or bone graft application for which the graft will be used. FIG. 3 shows a cross section of a calcaneus portion 18 cut from a donor, and FIG. 4 shows and end view of the bone shown in FIG. 3. As shown in FIG. 3, the bone comprises substantially cancellous bone 20 at least partially surrounded by a thin layer of cortical bone 22.

According to one or more embodiments, after a calcaneus portion 18 has been cut along line 21, each calcaneus portion 18 having a distal part 18B and a proximal part 18A will be cut and processed further, depending on the specific procedure in which the allograft material will be used. As used herein, when referring to bones or other body parts, the term “proximal” means closest to the heart and the term “distal” means more distant from the heart. For the calcaneus, the proximal part refers the part closest to the top of the foot, and the distal part refers to the part closest to the bottom of the foot. When referring to tools and instruments, the term “proximal” means closest to the practitioner and the term “distal” means distant from the practitioner.

In ALIF procedures, the proximal part 18B may be cut from the calcaneus portion 18, and the distal part 18B may be used as an implant in an ALIF procedure. For other types of interbody lumbar fusions, for example, PLIF and TLIF procedures, it may be desirable to further cut the distal part 18B into two, three, four or more subsections as shown by the dotted lines 24 in FIG. 5. According to certain embodiments, when the implant is placed in the lumbar or lumbosacral spine, the proximal or top portion of the implant faces the anterior aspect of the spine. In other embodiments, when the implant is used in the thoracic spine, the proximal portion of the implant faces the posterior aspect of the spine.

Of course, the above description provides a few limited examples of the types and number of cuts that may be used in processing calcaneus according to one or more embodiments of the present invention. It is within the scope of the invention to cut the calcaneus in different ways.

The cut bone is stored in an appropriate manner as is known, and is subsequently prepared for implantation in accordance with a fusion or bone graft procedure, including, spinal interbody fusion, particularly in the lumbar spine. A surgeon surgically implants the calcaneus graft between two vertebrae in a manner to allow for tissue or bone growth between the implant and the adjacent vertebrae. Bone inducing substances may be used together with the graft to induce, instigate or speed tissue or bone growth. Bone morphogenic proteins (BMPs), such as OP-1 offered by Stryker, bone graft materials made up in whole or in part of bone chips and tissue, bone growth factors, and bone growth hormones can be packed around or adjacent the graft between the vertebrae or coated on or around the implant. Such bone inducing substances may be naturally occurring, synthetic, or a combination of these.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims and their equivalents.

1. A lumbar or thoracic spinal implant comprising:
a graft comprised of a solid body of substantially cancellous bone sized and shaped for placement between two lumbar vertebral bodies, wherein the implant is capable of bearing weight in the lumbar or thoracic spine of a human being.

2. The implant of claim 1, wherein the graft is an allograft.

3. The implant of claim 2, wherein the allograft is comprised of at least about 60% cancellous bone by volume.

4. The implant of claim 2, wherein the allograft is comprised of at least about 80% cancellous bone by volume.

5. The implant of claim 2, wherein the allograft is comprised of at least about 95% cancellous bone by volume.

6. The implant of claim 2, wherein the allograft is comprised of at least about 98% cancellous bone by volume.

7. The implant of claim 6, wherein the surface area of the implant in contact with the vertebral bodies comprises at least 98% cancellous bone.

8. The implant of claim 1, wherein the bone source comprises calcaneus.

9. The implant of claim 7, wherein the contact surface area of the implant is greater than about 6 square centimeters.

10. The implant of claim 1, wherein the implant is capable of bearing a load of greater than about 500 pounds.

11. A lumbar spinal implant comprising an allograft comprised of calcaneus.

12. The spinal implant of claim 11, wherein the allograft is in the form of a solid body.

13. The spinal implant of claim 12, wherein allograft is sized and shaped for placement between two lumbar or thoracic vertebral bodies and capable of bearing weight in the lumbar spine of a human being.

14. The spinal implant of claim 13, wherein the allograft is capable of bearing 1000 pounds.

15. The spinal implant of claim 14, wherein the implant comprises a thin cortex partially surrounding a core of cancellous bone.

16. The spinal implant of claim 15, wherein the surface area in contact between the intervertebral bodies and the implant comprises at least about 80% cancellous bone.

17. The spinal implant of claim 15, wherein the contact surface area between the intervertebral bodies and the implant comprises at least about 98% cancellous bone.

18. The spinal implant of claim 11, wherein the implant has a thickness between about 0.5 centimeters and 5 centimeters.

19. A method of performing a spinal fusion on a patient comprising:
implanting a graft comprising substantially cancellous bone between lumbar or thoracic vertebral bodies.

20. The method of claim 19, graft is an allograft.

21. The method of claim 20, wherein the allograft comprises calcaneus.

22. The method of claim 20, wherein the surface area in contact between the allograft and the vertebral bodies is at least about 6 square centimeters.

23. The method of claim 20, wherein the allograft has a weight bearing capacity of at least about 1000 pounds.

24. A lumbar or thoracic spinal implant comprising:
an allograft comprised of a solid body of bone comprised of at least about 10% cancellous bone by volume sized and shaped for placement between two lumbar or thoracic vertebral bodies, wherein the implant is capable of bearing weight in the lumbar or thoracic spine of a human being.

25. The implant of claim 23, wherein the allograft is capable of bearing 1000 pounds.

26. A method of making an allograft lumbar or thoracic spine implant comprising:
cutting a portion of the calcaneus from a donor in a size and shape for insertion between two vertebral bodies.

27. The method of claim 25, wherein a plurality of cross sections is cut substantially perpendicular to the long axis and from a central portion of the calcaneus.

28. The method of claim 26, further comprising cutting at least a portion from one of the cross sections to provide an implant for use in a anterior interbody lumbar fusion procedure.

29. The method of claim 27, comprising cutting the portion to provide two or more subsections to provide an implant for use in a transforaminal lumbar interbody fusion or posterior interbody lumbar fusion.

30. A method of performing a spinal fusion on a patient comprising:
obtaining a graft comprised of substantially cancellous bone;
storing the graft; and
implanting the graft in the lumbar or thoracic spine.
